(19) United States
(12) Patent Application Publication
(10) Pub. No.: US 2005/0031557 A1
(43) Pub. Date: Feb. 10, 2005

(54) Oral administration of beta-carotene, lycopene and lutein for human skin protection

(76) Inventors: Christine Gaertner, Duesseldorf (DE); Wilhelm Stahl, Duesseldorf (DE); Ulrike Heinrich, Wetter (DE)

Correspondence Address: COGNIS CORPORATION;PATENT DEPARTMENT, 300 BROOKSIDE AVENUE, AMBLER, PA, 19002, US

(21) Appl. No.: 10/936,319

(22) Filed: Sep. 8, 2004

Related U.S. Application Data
(63) Continuation of application No. 10/070,590, filed on Jul. 30, 2002, now abandoned.

(30) Foreign Application Priority Data
Sep. 8, 1999 (DE) 199 42 774.7

(51) Int. Cl. A61K 7/42; A61K 31/015

(52) U.S. Cl. 424/59; 514/763

(57) ABSTRACT

Methods of improving the sun protection factor of human skin and methods of inhibiting the aging of human skin via the oral administration of a composition comprising (a) β-carotene, (b) lutein and (c) lycopene, in a ratio by weight (a):(b):(c) of from 1:0.5:0.5 to 1:1.5:1.5 are described.

### FIELD OF THE INVENTION

This invention relates generally to orally administered sun protection preparations and more particularly to a mixture of β-carotene, lutein and lycopene.

### PRIOR ART

Under the influence of the sun's rays, normal skin is pigmented by the formation of melanin. Exposure to long-wave UV-A light results in darkening of the melanins already present in the epidermis without any harmful effects while exposure to short-wave UV-B radiation results in the formation of new melanin. However, before the protective pigment can be formed, the skin is exposed to the effect of unfiltered radiation which can lead to reddening of the skin (erythema), inflammation of the skin (sunburn) or even to blisters, depending on the exposure time. The strain on the organism associated with such skin lesions, for example in connection with the distribution of histamines, can additionally lead to headache, lassitude, fever, heart and circulation problems and the like. In addition, long-term exposure can lead to cumulative DNA damage which can result in skin cancer. Consumers seeking to protect themselves against the harmful effects of the sun basically have two choices: first, they can protect the skin by topical application of cosmetic preparations containing UV protection factors, second they can increase the skin's own protection factor by oral application of suitable compounds.

European patent application EP 0 712 630 A2 (JBC Cosmetics) describes an orally administered preparation containing a carotinoid, a tocopherol, ascorbic acid and selenium. This preparation is intended to tan the skin and to prevent sun allergies (photodermatoses). α-Carotene, β-carotene and lycopene are used as the carotinoids in daily doses of 60 to 150 mg.

French patent application FR 2 698 268 A1 (L'Oréal) describes a composition for oral administration to human beings which contains tyrosine and/or phenyl alanine, a copper salt and a mixture of vitamins. Carotenes, vitamin E, niacin and vitamin C may be used as the vitamins. The carotenes mentioned include α-, β- and γ-carotene and lycopene which may be used in doses of 5 to 50 mg. The preparation is intended to protect the skin against the harmful effects of UV radiation.

Sun protection preparations for topical application where synthetic UV filters are replaced by substances of natural origin are described in EP 0 747 039 A2 (SA.FO.SA.). These sun protection preparations contain a mixture of amino acids, vitamins and/or provitamins, nucleoderivatives and vegetable extracts and may be used in the form of gels, creams or oils.

International patent application WO 97/47278 (Laboratoires Oenobiol.) claims a mixture for oral application containing
(a) at least one natural carotinoid with provitamin A character (either α- or β-carotene),
(b) at least one natural carotinoid without provitamin A character (lycopene) and
(c) another carotinoid selected from the group consisting of zeaxanthin, cryptoxanthin and lutein,
   the ratio of (a) to (b) being 0.95:1 to 1:50. In Example 1, this application describes a composition of 2.86 mg β-carotene and 3 mg lycopene. The mixture also contains 0.07 mg lutein as a secondary component of the β-carotene source.

Accordingly, the prior art literature describes numerous oral preparations which are supposed to increase the skin's own sun protection factor. Most of these preparations are based on α- or β-carotene. Since studies have shown that the supplementation of β-carotene can increase the incidence of lung cancer (ATBC Study, The New England Journal of Medicine, 1994, 330, 1029-1035 and CARET Study, G. S. Omenn et al., The New England Journal of Medicine, 1996, 334, 1150-1155), there is a need for a substitute or partial substitute for β-carotene in known oral sun protection preparations.

Accordingly, the problem addressed by the present invention was to provide improved sun protection preparations for oral administration. More particularly, by comparison with known sun protection preparations, a proportion of α- or β-carotene would be replaced by other, at least equally effective substances. The requirements which these substitutes would be expected to satisfy would be stringent. Besides providing comparable or better protection against the sun, they would have to be toxicologically safe and easy to handle and formulate. In addition, the substitutes in question would preferably be substances of natural origin. Besides increasing the skin's own sun protection factor, the sun protection preparations provided by the invention would also delay ageing of the skin.

### DESCRIPTION OF THE INVENTION

The present invention relates to orally administered preparations containing
(a) β-carotene,
(b) lutein and
(c) lycopene
   in a ratio by weight of (a) to (b) to (c) of 1:(0.5-1.5):(0.5 -1.5) in a carrier suitable for oral administration.

It has surprisingly been found that the oral administration of the preparations according to the invention increases the sun protection factor of the skin and at the same time delays ageing of the skin. The mixtures are toxicologically safe for oral administration and are easy to formulate. It has surprisingly been found that the mixture of these three particular carotinoids in the claimed ratio to one another is particularly suitable for increasing the sun protection factor of the skin and for delaying the ageing process of the skin. In contrast to the mixtures of WO 97/47278, the mixtures according to the invention produce a distinctly improved increase in the sun protection factor of the skin.

β-Carotene

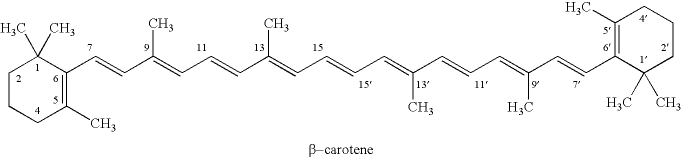

βCarotene is an 11x-unsaturated tetraterpene. The chemical skeleton consists of nine conjugated double bonds and two β-ionone ring structures at the ends of the molecule where the double bonds of the β-ionone system are in conjugation with the unsaturated system of the polyene chain. The double bonds may be in the trans position (trans-p-carotene, β,β-carotene, provitamin A) or in the cis position (for example 9-cis-β-carotene and 13-cis-β-carotene). β-carotene in the context of the invention encompasses both the cis and the trans isomers of β-carotene. The β-carotene may be obtained both by extraction from vegetable sources (for example carrots and other vegetables, palm oil) or from animal materials, bacteria and/or algae (more particularly from the alga Dunaliella salina) and microbiologically or synthetically via vitamin A (retinol). It is particularly preferred to use β-carotene obtained by extraction from algae, more particularly by extraction from the alga Dunaliella salina which is commercially obtainable as Betatene®.

Lutein

Lutein in the context of the present invention includes both lutein itself [═(3R,3′R,6′R)-β,ε-carotene-3,3′-diol; C₄₀H₅₆O₂; MW 568.85] and the fatty acid esters of lutein. Suitable fatty acid esters are the esters of palmitic acid, myristic acid, stearic acid, lauric acid and oleic acid, the esters being both mono- and diesters and mixed forms (for example lutein myristyl palmitate). 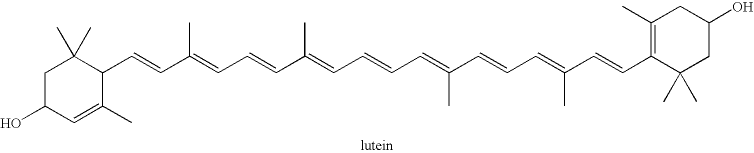
Lutein and its fatty acid esters may be obtained both by extraction from vegetable material (for example from varieties of Tagetes erecta (grass-of-Parnassus), stinging nettle leaves, lucern (for example alfalfa), palm oil), by extraction from animal material (for example egg yolk) and from bacteria or algae. It is particularly preferred to use lutein obtained by extraction from plants, more particularly lutein obtained by extraction from Tagetes erecta varieties which is commercially available as Xangold®.

Lycopene

Lycopene in the context of the present invention includes both the all trans isomer (ψ,ψ-carotene, C₄₀H₅₆, MW 536.85) and the cis isomers (such as, for example, 5-cis-, 9-cis-, 13-cis- and 15-cis-lycopene). Lycopene can be obtained by extraction from plants (tomato (Solanum lycopersicum), rose hip and other fruits, chanterelles (Cantharellus cibarius)) and by extraction from animal material. Lycopene can also be obtained by synthesis or extraction from microorganisms (fermentative protection). It is particularly preferred to use lycopene obtained by fermentation or by extraction from plants.

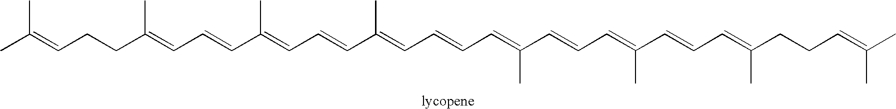

The ratio of the individual components to one another is crucial to the invention. It has surprisingly been found that, where components (a), (b) and (c) are present in a ratio of (a) to (b) to (c) of 1:(0.5-1.5):(0.5-1.5), the preparations obtained are distinguished from known preparations by a particularly effective increase in the sun protection factor of the skin. Particularly preferred preparations are those with a ratio of (a) to (b) to (c) of 1:(0.5-1.0):(0.5-1.0), more particularly 1.0:1.0:1.0, and a ratio of 1:0.5:0.5 and 1:0.75: 0.75.

Carriers Suitable for Oral Application

A key component of the preparations according to the invention is the carrier suitable for oral application. It is intended on the one hand to dissolve or disperse the carotinoid mixture according to the invention. In addition, it preferably supports the absorption of the carotinoids from the gastrointestinal tract. In principle, suitable carriers are any substances which perform these functions and which are toxicologically safe. Examples of suitable carriers are edible oils (particularly soybean oil), such as vegetable and fish oils which may optionally be partly hydrogenated, and carriers based on animal products, for example gelatine. Other suitable carrier materials are, for example, gum arabic, sucrose, lipids, mono- and diglycerides and maltodextrins. Where water is used as the carrier material, it is standard practice to use a suitable emulsifier (for example lecithins, sorbitan monolaurate).

The preparations may be orally administered, for example, as solutions, oils, emulsions, suspensions or dispersions. Suitable carrier forms are, for example, capsules or tablets. The preparations according to the invention are normally present in the form of soft gelatine capsules.

The preparations according to the invention are normally prepared by preparing a mixture of β-carotene, lutein and lycopene and then encapsulating the mixture thus prepared together with the carrier material.

The present invention includes the observation that the preparations according to the invention may be added to foods and that the foods thus enriched may be used as carriers for oral administration.

The present invention also includes the observation that typical antioxidants such as, for example, ascorbyl palmitate (E 304), mixed tocopherols (E 306), citric acid (E 330) or L-ascorbic acid (E 300) may be added to the preparations according to the invention.

In a preferred embodiment of the invention, the preparations according to the invention contain at least one other substance selected from the group consisting of α-carotene, astaxanthin, α-cryptoxanthin, β-cryptoxanthin, zeaxanthin, phytoene, phytofluene, γ-carotene and neurosporin.

The systematic names of the substances mentioned are as follows:

| | |
|---|---|
| α-carotene | β,ε-carotene |
| astaxanthin | (3S,3′S)-3,3′-dihydroxy-β,β-carotene-4,4′-dione |
| α-cryptoxanthin | (3R)-β,ε-carotene-3-ol |
| β-cryptoxanthin | (3R)-β,β-carotene-3-ol |
| zeaxanthin | (3R,3′R)-β,β-carotene-3,3′-diol) |
| phytoene | 7,8,11,12,7′,8′,11′,12′-octahydro-ψ,ψ-carotrene |
| phytofluene | 7,8,11,12,7′,8′-hexahydro-ψ,ψ-carotene |
| γ-carotene | β,ψ-carotene |
| neurosporin | 7,8-ψ,ψ-carotene. |

It is particularly preferred to use α-carotene.

It has surprisingly been found that the sun protection factor of the skin is increased by the oral administration of preparations containing (a) β-carotene, (b) lutein and (c) lycopene in a ratio by weight of (a) to (b) to (c) of 1:(0.5-1.5):(0.5-1.5) in a carrier suitable for oral administration. Accordingly, the present invention also relates to a process for increasing the sun protection factor of human skin, characterized in that the preparations according to the invention are orally administered.

The sun protection factor of the skin may be determined by any of the methods known to the expert such as, for example, determination of the minimum erythema activity (MED) as described by COLIPA. Other methods include determination of the melanin content and the concentration of the carotinoids in the skin by reflection spectrophotometry and/or HPLC and chromometric determination of skin color (a, b and L values). A description of these methods can be found, for example, in Biochemistry and Molecular Biology International, 42, No. 5, 1997, pp. 1023-1033.

The duration of supplementation is normally determined by the existing sun protection factor of the skin and by the—individually very different—absorption capacity. It may be carried out for several days, several weeks or even for several months or years. Because the preparations according to the invention are toxicologically safe, supplementation may even be carried out indefinitely, as desirable for example in people exposed much more than normal UV radiation.

It has surprisingly been found that the ageing process of human skin is delayed by the oral administration of preparations containing (a), (b) and (c) in a ratio by weight of 1:(0.5-1.5):(0.5-1.5) in a carrier suitable for oral administration. Accordingly, the present invention also relates to a process for delaying the ageing process of the skin, characterized in that the preparations according to the invention are orally administered.

The duration of supplementation is normally determined by the state of ageing of the skin and by the—individually very different—absorption capacity. It may be carried out for several days, several weeks or even for several months or years. Because the preparations according to the invention are toxicologically safe, supplementation may even be carried out indefinitely.

The quantity of components (a), (b) and (c)—expressed as a daily dose—is normally between 1 and 40 mg per component, with the proviso that the ratio of (a) to (b) to (c) is 1:(0.5-1.5):(0.5-1.5). Quantities of 2 to 25 mg per component and more particularly 5 to 10 mg per component are preferred. The preparations according to the invention may be administered as a single daily dose or as several doses distributed over a day.

The present invention also relates to the use of the orally administered preparations claimed in claim 1 for increasing the sun protection factor of human skin.

The present invention also relates to the use of the orally administered preparations claimed in claim 1 for delaying the ageing process of human skin.

### EXAMPLES

The carotinoid absorption and photoprotection studies were carried out using a panel of 36 volunteers with healthy skin of light type II (Fitzpatrick & Pathak test). The starting values for each volunteer were determined at the beginning of the 12-week study. An interim study was conducted after 6 weeks and the final study after 12 weeks. The volunteers were divided into three groups of twelve who received the following daily doses:
1st group: 25 mg Betatene® (corresponds to 24 mg β-carotene)
2nd group: 8.3 mg Betatene® (corresponds to 8 mg β-carotene), 8 mg lycopene, 8 mg lutein (Xangold®)
3rd group: placebo capsules.

The carotinoids were administered in soft gelatine capsules with 140 mg soybean oil.

The concentration of β-carotene, lycopene and lutein in the skin was determined by reflection spectrometry which was carried out on an area of 1 cm² of the forehead, the back of the hand, the palm of the hand, the inside of the lower arm and the back. The change in color of the skin during supplementation was differentiated by a Minolta chromameter (L, a, b system) into reddening of the skin (a values), yellow component (b values) and lightness of the skin (L values). The concentration of β-carotene, lycopene and lutein in the serum was determined by high-pressure liquid chromatography (HPLC).

The results are set out in Table 1 and represent the average values for the volunteer panel on completion of the study. The photoprotective value is expressed relative to the blank value (i.e. no addition of carotinoids, group 3).

TABLE 1
Photoprotective effect

| Group | Carotinoids (daily dose) | Photoprotection [%-rel.] |
|---|---|---|
| 1 | β-carotene (24 mg) | 200 |
| 2 | β-carotene (8 mg), lycopene (8 mg) | 270 |
| | and letein (8 mg) | |
| 3 | Control group with no carotinoids | 100 |

It can be seen that the administration of the Betatene mixture (β-carotene) doubles the photoprotection of the skin (group 2) in relation to the blank value (group 3). Where mixtures of Betatene (β-carotene) and lutein and lycopene are used, a distinct increase in photoprotection is obtained (group 2). The group 2 results clearly show that this effect is not an additive one because the same quantity of β-carotene (group 3) fails to achieve this protective effect.

1-7. (canceled).

8. A method of improving the sun protection factor of human skin, the method comprising:
(i) providing a composition comprising (a) β-carotene, (b) lutein and (c) lycopene, in a ratio by weight (a):(b):(c) of from 1:0.5:0.5 to 1:1.5:1.5; and
(ii) orally administering the composition to a human.

9. The method according to claim 8, wherein the composition further comprises one or more components selected from the group consisting of α-carotene, astaxanthin, α-cryptoxanthin, β-cryptoxanthin, zeaxanthin, phytoene, phtyofluene, γ-carotene and neurosporin.

10. The method according to claim 8, wherein the β-carotene, the lutein and the lycopene in a ratio by weight of from 1:0.5:0.5 to 1:1.0:1.0.

11. The method according to claim 8, wherein the composition is dispersed in an edible oil.

12. The method according to claim 8, wherein the β-carotene, the lutein and the lycopene are each present in an amount of from 1 to 40 mg.

13. A method of inhibiting the aging of human skin, the method comprising:
(i) providing a composition comprising (a) β-carotene, (b) lutein and (c) lycopene, in a ratio by weight (a):(b):(c) of from 1:0.5:0.5 to 1:1.5:1.5; and
(ii) orally administering the composition to a human.

14. The method according to claim 13, wherein the composition further comprises one or more components selected from the group consisting of α-carotene, astaxanthin, α-cryptoxanthin, β-cryptoxanthin, zeaxanthin, phytoene, phtyofluene, γ-carotene and neurosporin.

15. The method according to claim 13, wherein the β-carotene, the lutein and the lycopene in a ratio by weight of from 1:0.5:0.5 to 1:1.0:1.0.

16. The method according to claim 13, wherein the composition is dispersed in an edible oil.

17. The method according to claim 13, wherein the β-carotene, the lutein and the lycopene are each present in an amount of from 1 to 40 mg.
